(19) United States
(12) Patent Application Publication
(10) Pub. No.: US 2009/0131449 A1
(43) Pub. Date: May 21, 2009

(54) USE OF TRPV1 RECEPTOR ANTAGONISTS FOR TREATING DRY EYE AND OCULAR PAIN

(75) Inventors: John M. Yanni, Burleson, TX (US); Daniel A. Gamache, Arlington, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/273,047

(22) Filed: Nov. 18, 2008

Related U.S. Application Data
(60) Provisional application No. 60/988,901, filed on Nov. 19, 2007.

(51) Int. Cl. A61K 31/497

(52) U.S. Cl. 514/252.11; 514/255.05

(57) ABSTRACT

Methods of treating symptoms of dry eye by administering inhibitors of transient receptor potential cation channel, subfamily V, member 1 (TRPV1) are disclosed. Methods of preventing or alleviating ocular pain by administering TRPV1 inhibitors are also disclosed.

The present application claims the benefit of U.S. Provisional Patent Application Ser. No. 60/988,901, filed on Nov. 19, 2007, the disclosure of which is specifically incorporated by reference herein.

### FIELD OF THE INVENTION

The invention relates to the treatment of ocular pain and symptoms of dry eye disorders. In particular, the invention relates to the use of certain transient receptor potential cation channel, subfamily V, member 1 (TRPV1) inhibitors in the treatment of dry eye.

### BACKGROUND OF THE INVENTION

Pain is a perceived nociceptive response to local stimuli in the body. The perception of pain at the level of the central nervous system requires the transmission of painful stimuli by peripheral sensory nerve fibers. Upon stimulation of tissue (i.e., thermal, mechanical or chemical), electrochemical signals are transmitted from the sensory nerve endings to the spinal column, and hence to the brain where pain is perceived.

The cornea is highly innervated with sensory afferents which transmit various painful stimuli to the central nervous system. Pain conditions involving the eye, therefore, can arise in numerous instances, such as: foreign body stimulus, inflammation, dry eye syndrome, accidental trauma, surgical procedures and post-surgical recovery. For example, ocular pain can result from photorefractive keratotomy (“PRK”), a vision correcting, surgical procedure whereby a laser is used to shape the cornea. This process involves the photoablation of Bowman's membrane and the stromal levels of the cornea. As a result, the denuding of the nerve-containing epithelial layers of the cornea can cause some patients to experience pain following laser surgery until the epithelium regenerates.

Various therapies have been attempted for the alleviation of pain. The use of non-steroidal anti-inflammatory drugs (NSAIDs), such as diclofenac, have been developed to treat pain. These agents inhibit cyclooxygenase dependent prostaglandin synthesis. Prostaglandins can modulate pain perception at the level of the central nervous system and systemic administration of NSAIDs is known to provide analgesia. However, the use of NSAIDs can involve undesired side effects including gastrointestinal bleeding and kidney dysfunction.

Local anesthetics are another class of pain modulators that relieve pain by directly inhibiting nerve cellular function. One problem with local anesthetic therapy is that the anesthetics exhibit a short duration of action. Another problem with the use of local anesthetics is that their mechanism of action, non-specific membrane stabilization, can have the undesired coincident effect of also inhibiting biological functions of other cells, such as fibroblasts and surrounding neural cells. Therefore, even though pain sensation can be abated with local anesthetic treatment, healing and normal function of the tissue may be significantly compromised. There is a need, therefore, to discover agents which potently and specifically inhibit the transmission of painful stimuli by sensory afferents, without local anesthetic activity, following topical ocular application.

In addition to treating ocular pain, local topical ocular application of anesthetics has been proposed to reduce or eliminate sensations on the ocular surface to treat the symptoms of dry eye. However, chronic use of local anesthetics is accompanied by toxic side effects.

Dry eye, also referred to as keratoconjunctivitis sicca, is a common ophthalmological disorder affecting millions of persons each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease and cicatricial pemphigoid, may also lead to dry eye conditions. Transient symptoms of dry eye associated with refractive surgery have been reported to last in some cases from six weeks to six months or more following surgery.

Although it appears that dry eye may result from a number of unrelated pathogenic causes, all presentations of the complication share a common effect, that is the breakdown of the pre-ocular tear film, which results in exposure of the ocular surface, dehydration, and cytokine production resulting in many of the symptoms outlined above (Lemp, Report of the National Eye Institute/Industry Workshop on Clinical Trials in Dry Eyes, The CLAO Journal, volume 21, number 4, pages 221-231 (1995)).

Practitioners have taken several approaches to the treatment of dry eye. One common approach has been to supplement and stabilize the ocular tear film using so-called artificial tears instilled throughout the day. Other approaches include the use of ocular inserts that provide a tear substitute or stimulation of endogenous tear production.

Examples of the tear substitution approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids and oils. Phospholipid compositions have been shown to be useful in treating dry eye; see, e.g., McCulley and Shine, Tear film structure and dry eye, Contactologia, volume 20(4), pages 145-49 (1998); and Shine and McCulley, Keratoconjunctivitis sicca associated with meibomian secretion polar lipid abnormality, Archives of Ophthalmology, volume 116(7), pages 849-52 (1998).

Another approach involves the provision of lubricating substances in lieu of artificial tears. For example, U.S. Pat. No. 4,818,537 (Guo) discloses the use of a lubricating, liposome-based composition, and U.S. Pat. No. 5,800,807 (Hu et al.) discloses compositions containing glycerin and propylene glycol for treating dry eye.

Although these approaches have met with some success, problems in the treatment of dry eye nevertheless remain, since the use of tear substitutes, while temporarily effective, generally requires repeated application over the course of a patient's waking hours. It is not uncommon for a patient to have to apply artificial tear solution ten to twenty times over the course of the day. Such an undertaking is not only cumbersome and time consuming, but is also potentially very expensive.

Aside from efforts described above, which are directed primarily to the palliative alleviation of symptoms associated with dry eye, methods and compositions directed to treatment of the physiological conditions that cause such symptoms have also been pursued. For example, U.S. Pat. No. 5,041,434 (Lubkin) discloses the use of sex steroids, such as conjugated estrogens, to treat dry eye conditions in post-menopausal women; U.S. Pat. No. 5,290,572 (MacKeen) discloses the use of finely divided calcium ion compositions to stimulate pre-ocular tear film production.

Such efforts to treat the underlying causes of dry eye have focused on treating inflammation of the relevant ocular tissues and meibomian gland dysfunction. The use of various types of agents for such treatment of dry eye patients has been disclosed, including steroids (e.g., U.S. Pat. No. 5,958,912; Marsh et al., Topical nonpreserved methylprednisolone therapy for keratoconjunctivitis sicca in Sjogren syndrome, Ophthalmology, 106(4): 811-816 (1999); and Pflugfelder et al., U.S. Pat. No. 6,153,607), cytokine release inhibitors (Yanni, J. M.; et. al. WO 00/03705 A1), cyclosporine A (Tauber, J. Adv. Exp. Med. Biol. 1998, 438 (Lacrimal Gland, Tear Film, and Dry Eye Syndromes 2), 969), and mucosecretatogues, such as 15-HETE (Yanni et. al., U.S. Pat. No. 5,696,166).

Transient receptor potential cation channel, subfamily V, member 1 (TRPV1), also known as capsaicin receptor and vanilloid receptor 1 (VR1), is an ion channel belonging to the transient receptor potential (TRP) family. TRPV1 is a non-selective cation channel that can be activated by heat, protons, and vanilloid compounds (e.g. capsaicin). Activation of TRPV1 leads to the release of neurotransmitters, and results in pain and inflammation. TRPV1 antagonists, which can alleviate inflammation and pain caused by TRPV1 activation, fall into two major categories, including those that inhibit both capsaicin and proton activation, and those that inhibit capsaicin but not proton activation. Several such TRPV1 antagonists are known, as described by Roberts and Connor (2006, Recent Patents on CNS Drug Discovery 1:65-76). As discussed herein, TRPV1 antagonists can effectively reduce ocular pain and reduce symptoms of dry eye without causing anesthesia effects on the ocular surface.

### SUMMARY OF THE INVENTION

The invention provides methods for the treatment of dry eye symptoms, including symptoms of dry eye associated with refractive surgery such as LASIK surgery. According to the methods of the invention, certain TRPV1 antagonists are administered to a patient suffering from dry eye.

The invention also provides methods for the treatment of ocular pain and inflammation. According to the methods of the invention, TRPV1 antagonists are administered to a patient to prevent or alleviate pain in the eye.

The TRPV1 antagonists are preferably administered topically to the eye.

Specific preferred embodiments of the invention will become evident from the following more detailed description of certain preferred embodiments and the claims.

### DETAILED DESCRIPTION OF THE INVENTION

According to the invention, inhibitors of TRPV1 are administered to a patient suffering from dry eye. The compounds suitable for use in the present invention inhibit the activity of TRPV1 by binding to TRPV1 at the ocular surface of a patient, thereby reducing the pro-inflammatory effects of TRPV1 signaling associated with dry eye. The use of TRPV1 antagonists for treating dry eye provides an advantage over current therapies that involve anesthetics, because local treatment of TRPV1 antagonists will not cause loss of ocular sensations associated with anesthesia or have a central analgesic effect. As shown in the Examples herein, TRPV1 antagonists are beneficial in treating various ocular pain states and other conditions that have a neurogenic inflammatory component. In particular, TRPV1 antagonists can inhibit endogenous agonists acting on TRPV1 that provide a major contribution to certain ocular pain conditions. The Examples herein also show that TRPV1 antagonists have significant topical analgesic activity without topical anesthetic activity, thus making them very useful for treating symptoms of dry eye and for treating ocular pain.

According to the invention, TRPV1 antagonists are administered to a patient to prevent or ameliorate ocular pain associated with various stimuli. For example, the TRPV1 antagonists and compositions of the present invention may be used in treating pain arising from allergens, inflammation, trauma, dry eye, and/or foreign body sensation, such as from contact lenses and surgery. The compounds of the present invention may be used for the treatment of pain following ocular surgery, such as PRK surgery. With such treatment, the TRPV1 antagonists can be individually dosed, or in combination with other pharmaceutical agents such as by methods disclosed in U.S. Pat. Nos. 4,939,135 and 5,401,510 (Robertson et al.), the entire contents of which are incorporated herein by reference. The compounds will be utilized in a concentration effective to prevent or ameliorate ocular pain.

The term “TRPV1 antagonist” and “TRPV1 inhibitor” includes any agent that can inhibit the activity of TRPV1 (i.e. block TRPV1-mediated signaling cascade) at an ophthalmically relevant concentration. As used herein, an “ophthalmically relevant concentration” is less than 5.0% (w/v). TRPV1 antagonists useful in the methods of the invention include, but are not limited to, fused azabicyclic, heterocyclic, and amide compounds as described, for example, in U.S. Patent Application No. 2004/0157849, U.S. Patent Application No. 2004/0209884, U.S. Patent Application No. 2005/0113576, International Patent Application No. WO 05/016890, U.S. Patent Application No. 2004/0254188, U.S. Patent Application No. 2005/0043351, International Patent Application No. WO 05/040121, U.S. Patent Application No. 2005/0085512, and Gomtsyan et al., 2005, J. Med. Chem. 48:744-752; fused pyridine derivatives as described, for example, in U.S. Patent Application No. 2004/0138454; pyridyl piperazinyl ureas as described, for example, in Swanson et al., 2005, J. Med. Chem. 48:1857-1872 and U.S. Patent Application No. 2005/0049241, as well as AMG8163 (Bannon et al., 2005, 11^{th} World Congress on Pain) and BCTC (Sun et al., 2003, Chem. Lett. 13:3611-3616); 2-(Piperazine-1-yl)-1H-Benzimidazole; pyridazinylpiperazines; urea derivatives as describe, for example, in U.S. Patent Application No. 2005/0107388, U.S. Patent Application No. 2005/0187291, and U.S. Patent Application No. 2005/0154230, as well as A-425619 (El Kouhen et al., 2005, J. Pharmacol. Exp. Ther. 314:400-409); cinnamides, including SB-366791 (Gunthorpe et al., 2004, Neuropharmacology 46:133-149) and AMG 9810 (Gavva et al., 2005, J. Pharmacol. Exp. Ther. 313:474-484); each of which is incorporated by reference. Particular TRPV1 antagonists useful in the methods of the invention include AMG-517 and AMG-628 (Amgen Inc., Thousand Oaks, Calif.).

TRPV1 antagonists useful in the methods of the invention are also described, for example, in International Patent Application No. WO 2006065484; International Patent Application No. WO 2003070247; U.S. Patent Application No. US 2005080095; International Patent Application No. WO 2005007642; International Patent Application No. WO 2003080578; International Patent Application No. WO 2004007459; International Patent Application No. WO 2006063178; International Patent Application No. WO 2006062981; International Patent Application No. WO 2006065646; International Patent Application No. WO 2006122250; International Patent Application No. WO 2007050732; International Patent Application No. WO 2005077938; International Patent Application No. WO 2005077944; International Patent Application No. WO 2004014871; U.S. Patent Application No. US 2003195201; U.S. Patent Application No. US 2004152690; International Patent Application No. WO 2003099284; International Patent Application No. WO 2004072068; International Patent Application No. WO 2006044527; International Patent Application No. WO 2002016318; International Patent Application No. WO 2002016317; International Patent Application No. WO 2006098554; International Patent Application No. WO 2006101318; International Patent Application No. WO 2006101321; International Patent Application No. WO 2007063925; International Patent Application No. WO 2006033620; International Patent Application No. WO 2006038871; International Patent Application No. WO 2006068592; International Patent Application No. WO 2006068593; International Patent Application No. WO 2006068618; International Patent Application No. WO 2004089881; International Patent Application No. WO 2007073303; International Patent Application No. WO 2007091946; International Patent Application No. WO 2007091948; International Patent Application No. WO 2007091947; International Patent Application No. WO 2003014064; International Patent Application No. WO 2003055848; International Patent Application No. WO 2003055484; International Patent Application No. WO 2004072020; International Patent Application No. WO 2005040119; International Patent Application No. WO 2005044786; International Patent Application No. WO 2005044802; International Patent Application No. WO 2005103018; International Patent Application No. WO 2006080821; International Patent Application No. WO 2005003084; International Patent Application No. WO 2004035533; International Patent Application No. WO 2003066595; International Patent Application No. WO 2003074520; International Patent Application No. WO 2004002983; International Patent Application No. WO 2004011441; International Patent Application No. WO 2004029031; International Patent Application No. WO 2005009988; International Patent Application No. WO 2005009987; International Patent Application No. WO 2005012287; International Patent Application No. WO 2005030753; International Patent Application No. WO 2005030766; International Patent Application No. WO 2004103281; International Patent Application No. WO 2002072536; International Patent Application No. WO 2002090326; International Patent Application No. WO 2003053945; International Patent Application No. WO 2003068749; International Patent Application No. WO 2005016915; International Patent Application No. WO 2005016922; International Patent Application No. WO 2005063260; International Patent Application No. WO 2007042906; International Patent Application No. WO 2006122772; International Patent Application No. WO 2005105798; International Patent Application No. WO 2006105971; International Patent Application No. WO 2006122799; International Patent Application No. WO 2006122776; International Patent Application No. WO 2006122773; International Patent Application No. WO 2006122771; International Patent Application No. WO 2006122777; International Patent Application No. WO 2006122770; International Patent Application No. WO 2006136245; International Patent Application No. WO 2004069792; International Patent Application No. WO 2006058338; International Patent Application No. WO 2006102645; International Patent Application No. WO 2007109355; International Patent Application No. WO 2006006741; International Patent Application No. WO 2006006740; International Patent Application No. WO 2005014580; International Patent Application No. WO 2007090134; International Patent Application No. WO 2003097586; International Patent Application No. WO 2004046133; International Patent Application No. WO 2004099177; International Patent Application No. WO 2005028445; International Patent Application No. WO 2005049601; International Patent Application No. WO 2005049613; International Patent Application No. WO 2005051390; International Patent Application No. WO 2005080391; International Patent Application No. WO 2006100520; International Patent Application No. WO 2006120481; International Patent Application No. WO 2006038041; International Patent Application No. WO 2006122200; International Patent Application No. WO 2007010383; International Patent Application No. WO 2002008221; International Patent Application No. WO 2006007851; International Patent Application No. WO 2006029142; International Patent Application No. WO 2003062209; International Patent Application No. WO 2004055003; International Patent Application No. WO 2004055004; International Patent Application No. WO 2004056774; International Patent Application No. WO 2005007648; International Patent Application No. WO 2005007646; International Patent Application No. WO 2005007652; International Patent Application No. WO 2005009977; International Patent Application No. WO 2005009982; International Patent Application No. WO 2005009980; International Patent Application No. WO 2005023807; International Patent Application No. WO 2005087227; International Patent Application No. WO 2006026135; International Patent Application No. WO 2006042289; International Patent Application No. WO 2006071538; International Patent Application No. WO 2006078992; International Patent Application No. WO 2006081388; International Patent Application No. WO 2007047575; International Patent Application No. WO 2007047576; International Patent Application No. WO 2004033435; International Patent Application No. WO 2005121116; International Patent Application No. WO 2005120510; International Patent Application No. WO 2007065662; International Patent Application No. WO 2007065888; International Patent Application No. WO 2007065663; International Patent Application No. WO 2002076946; International Patent Application No. WO 2007054480; International Patent Application No. WO 2007054474; International Patent Application No. WO 2005095329; International Patent Application No. WO 2006016218; International Patent Application No. WO 2006051378; International Patent Application No. WO 2006095263; International Patent Application No. WO 2006097817; International Patent Application No. WO 2006103503; International Patent Application No. WO 2005123666; International Patent Application No. WO 2006045498; International Patent Application No. WO 2004058754; International Patent Application No. WO 2005032493; International Patent Application No. WO 2005066171; International Patent Application No. WO 2005046683; International Patent Application No. WO 2006093832; International Patent Application No. WO 2007100758; International Patent Application No. WO 2006024776; International Patent Application No. WO 2007010138; International Patent Application No. WO 2007010144; and International Patent Application No. WO 2007088277; the disclosure of each of which is incorporated herein by reference.

According to the methods of the present invention, a composition comprising one or more of the specified TRPV1 antagonists and a pharmaceutically acceptable carrier for topical ophthalmic administration or implantation into the conjunctival sac or anterior chamber of the eye is administered to a mammal in need thereof. The compositions are formulated in accordance with methods known in the art for the particular route of administration desired.

The compositions administered according to the present invention comprise a pharmaceutically effective amount of one or more of the specified TRPV1 antagonists. As used herein, a “pharmaceutically effective amount” refers to that amount of one or more TRPV1 antagonists that prevents or alleviates ocular pain and/or is sufficient to reduce or eliminate symptoms of dry eye. Preferably, compositions are intended to be administered topically to the eye in the form of eye drops or eye ointments, wherein the total amount of TRPV1 antagonist will be about 0.001 to 5.0% (w/v). Preferably, the amount of TRPV1 antagonists is about 0.01 to about 5.0% (w/v).

Preferably, the compositions administered according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions. Suspensions may be preferred for cytokine synthesis inhibitors which are sparingly soluble in water.

The compositions administered according to the present invention may also include various other ingredients, including but not limited to surfactants, tonicity agents, buffers, preservatives, co-solvents and viscosity building agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 6.0-7.5.

Topical ophthalmic products may also be packaged in multidose form. Preservatives may thus be required to prevent microbial contamination during use. Suitable preservatives include: chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 5.0% w/v. Unit dose compositions of the present invention will be sterile, but typically unpreserved. Such compositions, therefore, generally will not contain preservatives. The ophthalmic compositions of the present invention may also be provided preservative free and packaged in unit dose form.

The preferred compositions of the present invention are intended for administration to a human patient suffering from ocular pain or dry eye or symptoms of dry eye. Preferably, such compositions will be administered topically. In general, the doses used for the above described purposes will vary, but will be in an effective amount to reduce or eliminate ocular pain and/or eliminate or improve dry eye conditions. Generally, 1-2 drops of such compositions will be administered one or more times per day. For example, the composition can be administered 2 to 3 times a day or as directed by an eye care provider.

A representative eye drop formulation is provided in Table 1 below.

TABLE 1

| Ingredient | Amount (% w/v) |
|---|---|
| TRPV1 antagonist | 0.001-5.0 |
| Boric Acid | 0.25 |
| Sodium Chloride | 0.75 |
| Disodium Edetate | 0.01 |
| Polyquaternium-1 | 0.001 |
| NaOH/HCl | q.s., pH = 7.4 |
| Purified Water | q.s. 100% |

The above composition is prepared by the following method. The batch quantities of boric acid, sodium chloride, disodium edetate, and polyquaternium-1 are weighed and dissolved by stirring in 90% of the batch quantity of purified water. The pH is adjusted to 7.4.±.0.1 with NaOH and/or HCl. The batch quantity of the TRPV1 antagonist as a stock solution is measured and added. Purified water is added to q.s. to 100%. The mixture is stirred for five minutes to homogenize and then filtered through a sterilizing filter membrane into a sterile recipient.

All references cited in this application are expressly incorporated by reference herein for any purpose.

Unless otherwise required by context, singular terms used herein shall include pluralities and plural terms shall include the singular.

### EXAMPLES

The following examples, including the experiments conducted and results achieved are provided for illustrative purposes only and are not to be construed as limiting the invention.

### Example 1

### TRPV1 Antagonists Reduce Ocular Pain

The effects of two transient receptor potential vanilloid receptor subfamily, member 1 (TRPV1) antagonists on ocular pain in rats were tested using a formalin-induced blink response assay. Sprague-Dawley rats were treated topical ocular with 20 μL of vehicle (maxidex vehicle), N-{4-[6-(4-trifluoromethyl-phenyl)-pyrimidin-4-yloxy]-benzothiazol-2-yl}-acetamide (AL-49975, also known as AMG-517, Amgen Inc., Thousand Oaks, Calif.), or (R)—N-(4-(6-(4-(1-(4-fluorophenyl)ethyl)piperazin-1-yl)pyrimidin-4-yloxy)benzo[d]thiazol-2-yl)acetamide (AL-49976, also known as AMG-628, Amgen Inc., Thousand Oaks, Calif.) to one eye only. After the appropriate pretreatment time of about 5 minutes, 5 μL of 0.1% formalin was applied topical ocular. Each rat was placed in a clear plastic box, and the number of blinks was counted for 1 minute immediately following the formalin challenge.

The results of the blink response assay indicated that AL-49975 inhibited the formalin-induced blink response in a dose-dependent fashion, achieving significant inhibition at the highest concentration tested (Table 1), and that AL-49976 also significantly inhibited the pain response at the highest concentration tested (Table 1).

TABLE 1
Effect of TRPV1 antagonists on Formalin-Induced Blink Response in Rats

| | | % | Pretreatment | Number of Blinks/1 min | % |
|---|---|---|---|---|---|
| Compound | ID | Conc. | Time (min) | Mean ± S.D. | Inhibition |
| vehicle | | | 5 | 72 ± 16 | |
| AL-49975 | Amgen | 1 | 5 | 44 ± 17 | 39* |
| AL-49975 | AMG-517 | 0.1 | 5 | 51 ± 18 | 29 |
| AL-49975 | TRPV1 antagonist | 0.01 | 5 | 58 ± 11 | 20 |
| | N-[4-[6-[4(Trifluoromethyl)phenyl)pyrimidin- | | | | |
| | 4-yloxy]benzothiazol-2-yl]acetamide | | | | |
| AL-49976 | Amgen | 1 | 5 | 47 ± 11 | 35* |
| AL-49976 | AMG-628 | 0.1 | 5 | 66 ± 23 | 9 |
| AL-49976 | TRPV1 antagonist | 0.01 | 5 | 58 ± 7 | 19 |
| | N-[4-[6-[4-[1(R)-(4-Fluorophenyl)ethyl]piperazin- | | | | |
| | 1-yl]pyrimidin-4-yloxy]benzothiazol-2-yl] | | | | |

*p < 0.05, Dunnett's t-test

### Example 2

### TRPV1 Antagonists do not have Topical Anesthetic Activities

Corneal anesthetic effects of TRPV1 antagonists were examined by analyzing suppression of blinks induced by mechanical touch. A Cochet-Bonnet Esthesiometer was used to determine corneal anesthetic activities of the TRPV1 antagonist, AMG-517 (AL-49975), in normal rats.

Male Sprague Dawley rats (˜500 g) were divided into groups of 6 each, restrained in a DecapiCone rat restraint, and secured at the posterior with tape. A hole was cut into the cone to expose the right eye. Twenty-four hours prior to the experiment the eyelashes and whiskers were trimmed with scissors. The right eye was dosed with 20 μl of drug or maxidex vehicle, and the timer was set for 5 minutes to allow the rat time to acclimate. The Cochet-Bonnet Esthesiometer fiber was set at 30 mm and perpendicularly touched by a masked observer to the center of the cornea 10 times with a 3 second delay between counts. Blinks were counted with each touch of the fiber, and a total score out of 10 was recorded. If more than one blink occurred in response to a single touch, this event was counted as one blink response.

The topical anesthetic, 0.5% proparacaine (Alcaine), inhibited the mechanical blink response by 95%, providing a reference for the study (Table 2). AL-49975 did not significantly inhibit the blink response at a 1% concentration. Thus, the TRPV1 antagonist, AMG-517, which displayed significant topical analgesic activity as a 1% suspension, did not have topical anesthetic activity at that same concentration.

TABLE 2

| | | | Total number | |
|---|---|---|---|---|
| | | | of Blinks/ | |
| | | Pretreatment | Total number | % |
| Compound | ID | Time (min) | of Touches | Blocked |
| vehicle | | 5 | 60/60 | 0 |
| 0.5% Alcaine | Amgen | 5 | 2/60 | 97* |
| 1% AL-49975 | AMG517 | 5 | 54/60 | 10 |

Note:
N = 6/group
*p < 0.01, Chi-squared vs. Vehicle

It should be understood that the foregoing disclosure emphasizes certain specific embodiments of the invention and that all modifications or alternatives equivalent thereto are within the spirit and scope of the invention as set forth in the appended claims.

What is claimed is:

1. A method for treating symptoms of dry eye which comprises administering to a mammal a composition comprising a pharmaceutically acceptable carrier and a pharmaceutically effective amount of a TRPV1 antagonist.

2. The method of claim 1 wherein the pharmaceutically effective amount of the TRPV1 antagonist is 0.001-5.0% (w/v).

3. The method of claim 1 wherein the pharmaceutically effective amount of the TRPV1 antagonist is 0.01-5.0% (w/v).

4. The method of claim 1 wherein the composition is topically administered to the eye.

5. The method of claim 1 wherein the dry eye is associated with refractive surgery.

6. The method of claim 1, wherein the TRPV1 antagonist is AMG-517 or AMG-628.

7. A method for the treatment of ocular pain which comprises administering to a mammal a composition comprising a pharmaceutically acceptable carrier and a pharmaceutically effective amount of a TRPV1 antagonist.

8. The method of claim 1 wherein the pharmaceutically effective amount of the TRPV1 antagonist is 0.001-5.0% (w/v).

9. The method of claim 1 wherein the pharmaceutically effective amount of the TRPV1 antagonist is 0.01-5.0% (w/v).

10. The method of claim 1 wherein the composition is topically administered to the eye.

11. The method of claim 1 wherein the pain is associated with refractive surgery.

12. The method of claim 1, wherein the TRPV1 antagonist is AMG-517 or AMG-628.
